(19) United States
(12) Patent Application Publication
(10) Pub. No.: US 2025/0018214 A1
(43) Pub. Date: Jan. 16, 2025

(54) INTRAORAL PHOTOTHERAPY DEVICE

(71) Applicant: TAIWAN REDEYE BIOMEDICAL INC., HSINCHU CITY (TW)

(72) Inventors: SHUO-TING YAN, HSINCHU CITY (TW); CHIEN-CHUNG TUAN, HSINCHU CITY (TW); TSUNG-JUI LIN, HSINCHU CITY (TW)

(21) Appl. No.: 18/351,621

(22) Filed: Jul. 13, 2023

(51) Int. Cl. A61N 5/06

(52) U.S. Cl. CPC A61N 5/0603; A61N 2005/0606; A61N 2005/0643; A61N 2005/0659; A61N 2005/0663

(57) ABSTRACT

An intraoral phototherapy device includes a transparent shell, a supporting board, and a flexible circuit board. The transparent shell includes a bite portion and a light portion. The bite portion is mounted on a light surface of the light portion. The supporting board is mounted in the light portion. The flexible circuit board is mounted on the supporting board, and includes at least one light emitting unit. The at least one light emitting unit emits a light having wavelength of 600-1000 nanometer (nm) toward the light surface. When a user bites the bite portion of the transparent shell, the light emitted by the at least one light emitting unit emits to a gum of the user. Therefore, the intraoral phototherapy device can stimulate an affected part of a gingivitis through a photobiomodulation therapy to achieve an effect of anti-inflammation or alleviating pain.

### BACKGROUND OF THE INVENTION

### 1. Field of the Invention

The present invention relates to a phototherapy device, especially an intraoral phototherapy device.

### 2. Description of the Related Art

Gingivitis causes symptoms such as redness and swelling of gums, and causes discomfort to teeth. Severe gingivitis may damage tissues around the teeth, which can lead to periodontitis, causing gum recession and tooth loss. Usually, mild gingivitis can be improved by brushing teeth and keeping mouth clean, and the mild gingivitis will heal naturally in about 10-14 days.

However, only brushing teeth or maintaining mouth clean requires a long recovery time for curing gingivitis, and the gums still have redness and swelling during the recovery time, causing discomfort to the teeth. If a patient seeks clinical help, the dentist usually prescribes anti-inflammatory drugs or painkillers for treatment, but the drugs may have adverse effects on human bodies. Therefore, conventional methods for curing gingivitis of brushing teeth, keeping mouth clean, or taking medication must be improved.

### SUMMARY OF THE INVENTION

In view of the above problems, the present invention provides an intraoral phototherapy device, which can promote healing of gingivitis through the Photobiomodulation therapy, and can have anti-inflammatory or analgesic effects. Therefore, questions of adverse effects or long recovery time caused by drug treatment can be avoided.

The intraoral phototherapy device includes a transparent shell, a supporting board, and a flexible circuit board.

The transparent shell includes a bite portion and a light portion. The light portion includes a light surface and a back surface. The bite portion is mounted on the light surface of the light portion.

The supporting board is mounted in the light portion of the transparent shell, and includes a circuit mounting surface. The circuit mounting surface faces to the light surface of the light portion.

The flexible circuit board is mounted on the circuit mounting surface of the supporting board, and includes a light emitting surface. The light emitting surface faces to the light surface of the light portion.

The flexible circuit board further includes at least one light emitting unit. The at least one light emitting unit is mounted on the light emitting surface, and the at least one light emitting unit emits a light having wavelength of 600-1000 nanometers (nm).

Adenosine triphosphate (ATP) is an important molecule that provides cellular energy, and can be regarded as energy currency of human body cells. The photobiomodulation therapy uses red light or near-infrared light to irradiate mitochondria in cells. When energy of the red light or the near-infrared light is absorbed by cytochrome C oxidase in the mitochondria, the Electron transport chain in the mitochondria can be accelerated to generate more ATP for providing to the cells, such that pain can be relieved, inflammation can reduced, wound healing can be promoted, and blood circulation can be promoted.

The photobiomodulation therapy has anti-inflammatory or pain-relieving effects, and can promote wound healing without causing damage to the human body. Therefore, when the gums are inflamed, the photobiomodulation therapy can help relieve the pain and discomfort caused by the gingivitis.

The present invention stimulates an affected part of the gingivitis through the photobiomodulation therapy, and light emitted by the at least one light emitting unit stimulates the gum tissue to achieve an effect of repairing or alleviating pain.

When the present invention is in use, a user only needs to bite the bite portion of the transparent shell, so that the light emitted by the at least one light emitting unit mounted on the flexible circuit board can irradiate the gums to relieve inflammation and pain of the gums. Then, sore gums, gums atrophy, wound healing, and other discomforts can be soothed.

In addition, the user only needs to bite the intraoral phototherapy device of the present invention, which is a non-invasive treatment, such that the discomfort of the user can be reduced, and medication is not required. Therefore, adverse effects of the medication on the human bodies can be also avoided.

### BRIEF DESCRIPTION OF THE DRAWINGS

FIG. 1 is a schematic diagram of an intraoral phototherapy device of the present invention;

FIG. 2 is a schematic perspective diagram of the intraoral phototherapy device of the present invention;

FIG. 3 is a schematic cross sectional diagram of the intraoral phototherapy device of the present invention;

FIG. 4 is a schematic diagram of a use state of the intraoral phototherapy device of the present invention;

FIG. 5 is another schematic diagram of the intraoral phototherapy device of the present invention;

FIG. 6 is a schematic side diagram of the intraoral phototherapy device of the present invention;

FIG. 7 is a schematic diagram of the appearances of a first embodiment of the intraoral phototherapy device of the present invention;

FIG. 8 is a block diagram of the first embodiment of the intraoral phototherapy device of the present invention;

FIG. 9 is a block diagram of a second embodiment of the intraoral phototherapy device of the present invention.

### DETAILED DESCRIPTION OF THE INVENTION

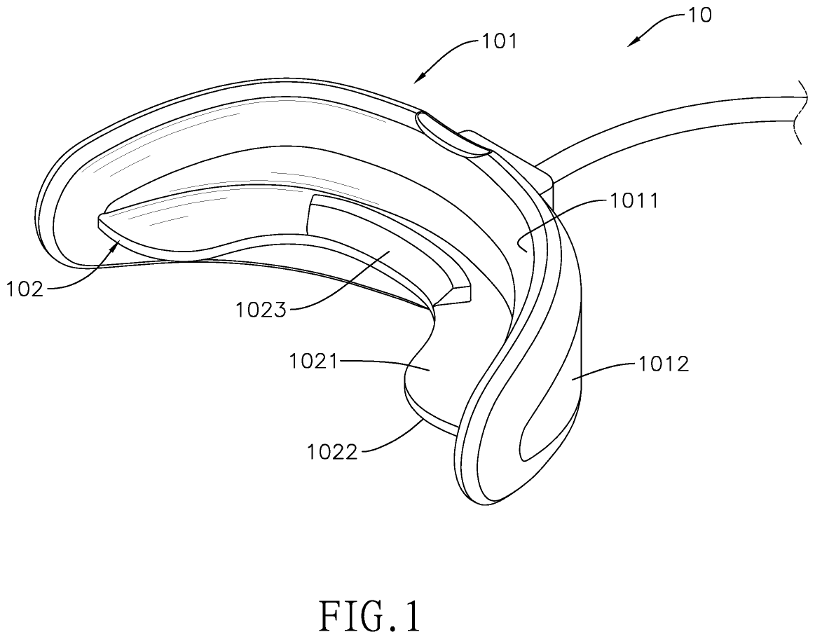

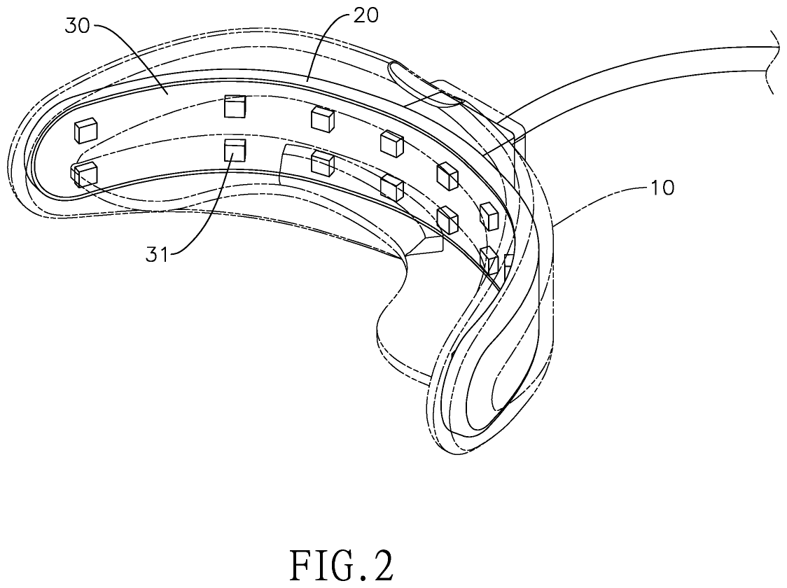

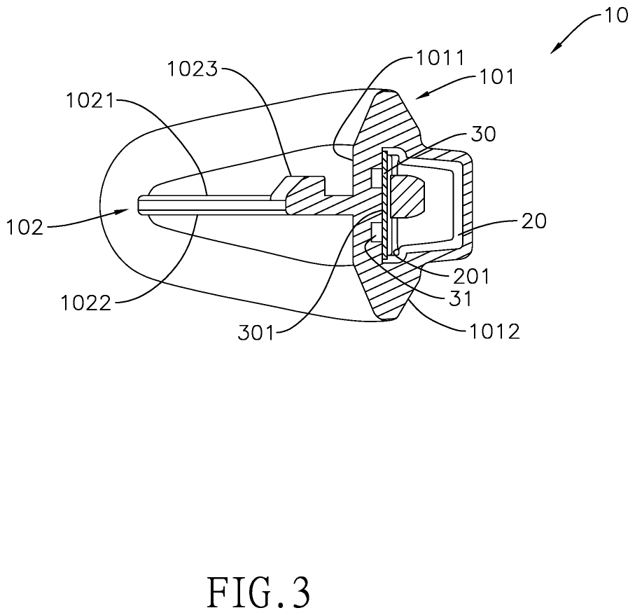

With reference to FIGS. 1 to 3, the present invention is an intraoral phototherapy device, and the intraoral phototherapy device includes a transparent shell 10, a supporting board 20, and a flexible circuit board 30.

The transparent shell 10 includes a light portion 101 and a bite portion 102. The light portion 101 includes a light surface 1011 and a back surface 1012. The bite portion 102 is mounted on the light surface 1011 of the light portion 101.

The supporting board 20 is mounted in the light portion 101 of the transparent shell 10, and includes a circuit mounting surface 201. The circuit mounting surface 201 faces to the light surface 1011 of the light portion 101.

The flexible circuit board 30 is mounted on the circuit mounting surface 201 of the supporting board 20, and includes a light emitting surface 301. The light emitting surface 301 faces to the light surface 1011 of the light portion 101.

The flexible circuit board 30 further includes at least one light emitting unit 31. The at least one light emitting unit 31 is mounted on the light emitting surface 301 of the flexible circuit board 30, and the at least one light emitting unit 31 emits a light having wavelength of 600-1000 nanometers (nm). In this embodiment, the light emitting unit 31 is a laser unit, a red light emitting unit (LED), or an infrared LED unit.

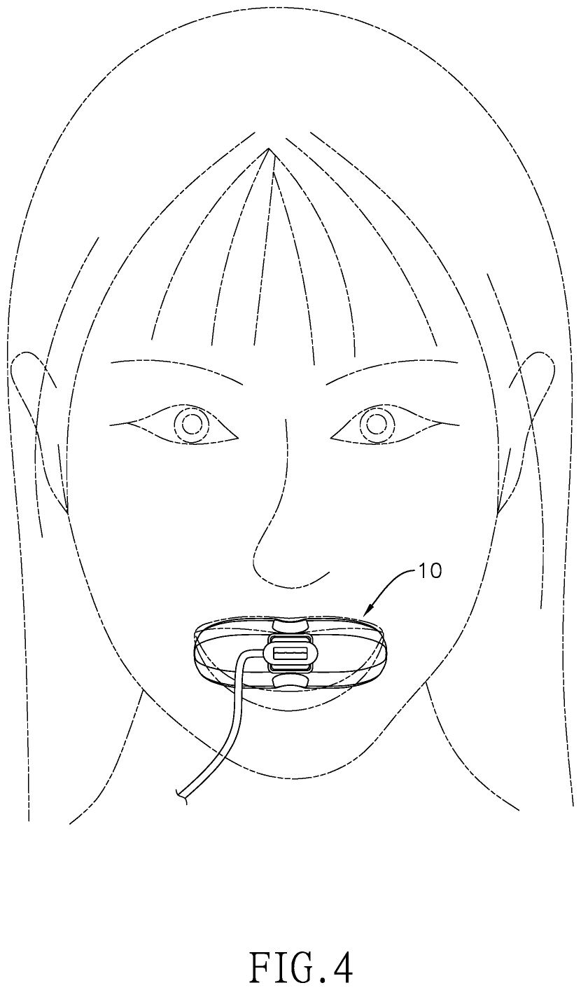

With reference to FIG. 4, since the bite portion 102 is mounted on the light surface 1011 of the light portion 101, when a user bites the bite portion 102, the light surface 1011 of the light portion 101 can directly face to a gum of the user. Therefore, when the user bites the bite portion 102 to use the intraoral phototherapy device, the at least one light emitting unit 31 mounted on the flexible circuit board 30 can directly be emitted to the gum of the user, such that sore gums, gums atrophy, wound healing, and other discomforts of the user can be soothed.

In this embodiment, the transparent shell 10 is made of a soft material. For example, the transparent shell 10 is made of silicone rubber, so that the user can directly bite the transparent shell 10, and the silicone rubber can provide a more comfortable feel of bite. At the same time, the silicone rubber has a waterproof function, such that the flexible circuit board 30 mounted in the transparent shell 10 can be protected from moisture.

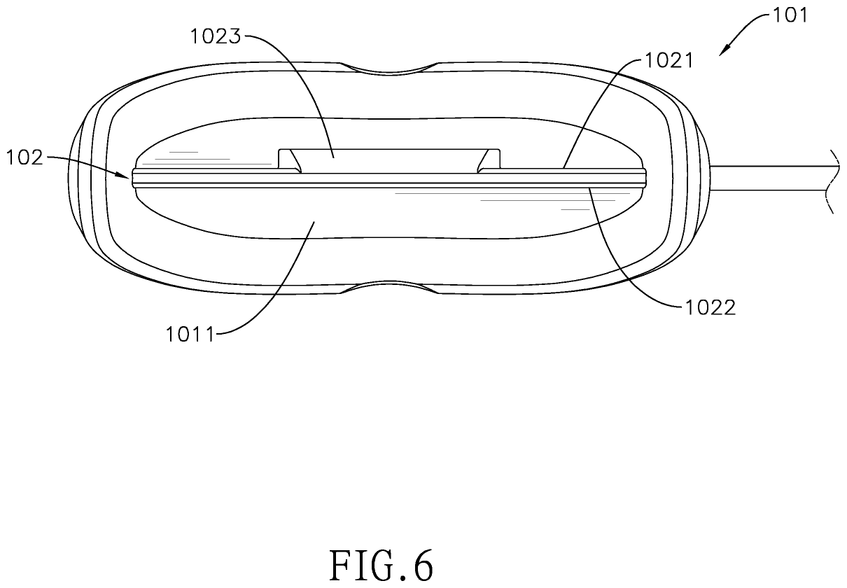

With reference to FIGS. 1, 3, 5, and 6, the bite portion 102 of the transparent shell 10 is flat, and has a first surface 1021 and a second surface 1022 opposite to the first surface 1021. The first surface 1021 and the second surface 1022 of the bite portion 102 are respectively perpendicular to the light surface 1011 of the light portion 101.

Therefore, the user can directly bite the flat bite portion 102 with teeth, and the bite portion 102 can allow the user to easily bite for improving comfort. Since the first surface 1021 and the second surface 1022 are respectively perpendicular to the light surface 1011 of the light portion 101, when the user bites the bite portion 102, the light surface 1011 of the light portion 101 can directly face to the gum of the user.

Moreover, the first surface 1021 of the bite portion 102 protrudes a positioning block 1023. When the user bites the bite portion 102, the teeth of the user can be positioned between the positioning block 1023 and the light surface 1011 of the light portion 101, such that the light surface 1011 of the light portion 101 can fittingly contact the gum of the user.

Furthermore, the light portion 101 of the transparent shell 10 is an arc plate. The light surface 1011 is opposite to the back surface 1012, and the light surface 1011 is an inner arc surface of the light portion 101. Since the light portion 101 is the arc plate, when the user bites the bite portion 102, the light surface 1011 of the light portion 101 can more fittingly contact the gum of the user according to the same shape.

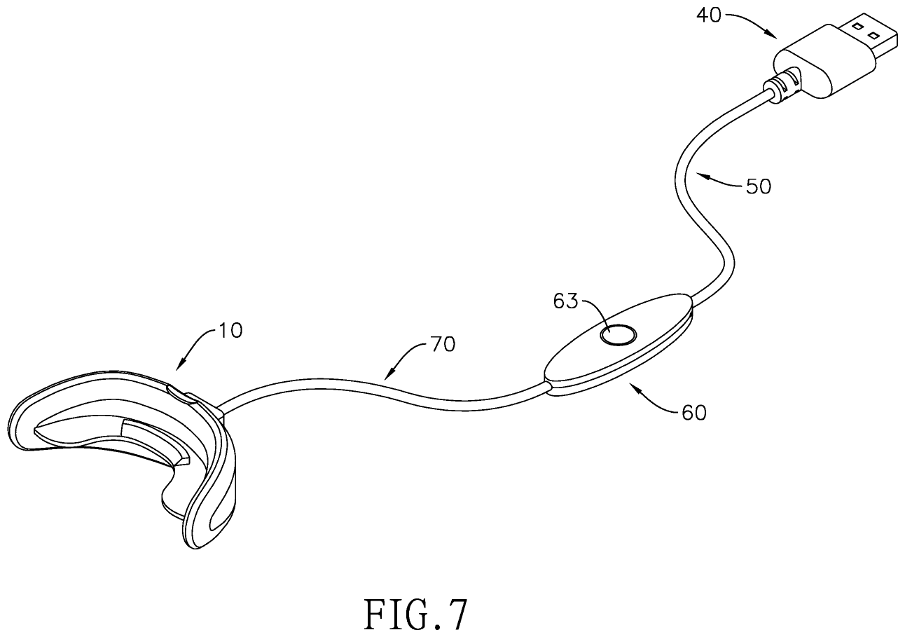

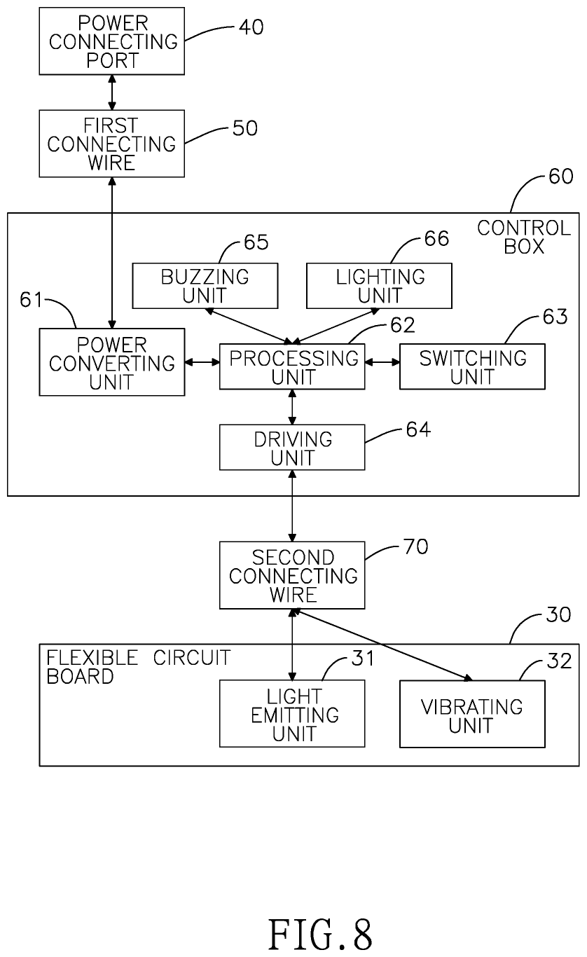

With reference to FIGS. 7 and 8, in a first embodiment of the present invention, the intraoral phototherapy device further includes a power connecting port 40, a first connecting wire 50, a control box 60, and a second connecting wire 70.

The control box 60 includes a power converting unit 61, a processing unit 62, a switching unit 63, a driving unit 64, a buzzing unit 65, and a lighting unit 66.

The power converting unit 61 connects to the power connecting port 40 through the first connecting wire 50. The power converting unit 61 receives an external electricity provided by an external power, and converts the external electricity to a powering electricity. In this embodiment, the power connecting port 40 is a universal serial bus (USB) male connecting port for connecting to a power adaptor, and the power connecting port 40 receives the external electricity with 5 volts (V) and 1 ampere (A).

The processing unit 62 is electrically connected to the power converting unit 61 for receiving the powering electricity.

The switching unit 63 is electrically connected to the processing unit 62. When the switching unit 63 generates a starting signal, the processing unit 62 receives the starting signal, and generates a light controlling signal according to the starting signal.

The driving unit 64 is electrically connected to the processing unit 62. When the driving unit 64 receives the light controlling signal, the driving unit 64 generates a light driving signal according to the light controlling signal.

The buzzing unit 65 is electrically connected to the processing unit 62. When the switching unit 63 generates the starting signal, the processing unit 62 receives the starting signal, and further generates a reminding signal according to the starting signal. When the buzzing unit 65 receives the reminding signal, the buzzing unit 65 generates a reminding audio according to the reminding signal.

The lighting unit 66 is electrically connected to the processing 62. When the switching unit 63 generates the starting signal, the processing unit 62 receives the starting signal, and further generates the reminding signal according to the starting signal. When the lighting unit 66 receives the reminding signal, the lighting unit 66 emits a reminding light according to the reminding signal. In this embodiment, the switching unit 63 is a circular push button, and the lighting unit 66 is an annular light emitting diode (LED) light around the switching unit 63. For example, the lighting unit 66 is a RGB LED. When the switching unit 66 is started by being pressed, the lighting unit 66 can emit a colorful light for reminding the user of a current state of the intraoral phototherapy device.

The second connecting wire 70 is electrically connected between the at least one light emitting unit 31 mounted on the flexible circuit board 30 and the driving unit 64 of the control box 60.

When the driving unit 64 generates the light driving signal, the at least one light emitting unit 31 receives the light driving signal through the second connecting wire, and emits the light according to the light driving signal.

Moreover, the flexible circuit board 30 further includes at least one vibrating unit 32, and the at least one vibrating unit 32 is mounted on the light emitting surface 301 of the flexible circuit board 30. When the switching unit 63 generates the starting signal, the processing unit 62 receives the starting signal, and further generates a vibration controlling signal according to the starting signal. When the driving unit 64 receives the vibration controlling signal, the driving unit 64 generates a vibration driving signal according to the vibration controlling signal. When the driving unit 64 generates the vibration driving signal, the at least one vibrating unit 32 receives the vibration driving signal through the second connecting wire 70, and generates vibrations according to the vibration driving signal.

Therefore, when the user uses the intraoral phototherapy device, not only the at least one light emitting unit 31 can emit the light to the gum of the user for soothing sore gums, gums atrophy, wound healing, and other discomforts of the user, but also the at least one vibrating unit 32 can generate vibration for providing an effect of massage to further sooth discomforts of the gum of the user.

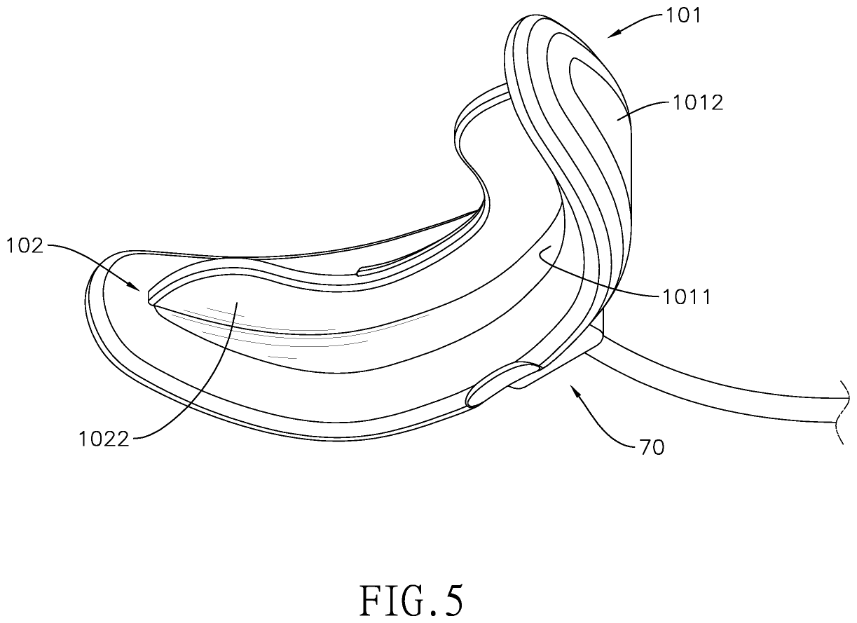

With reference to FIGS. 5 and 7, the second connecting wire 70 extends from the back surface 1012 of the light portion 101 into the transparent shell 10 to electrically connect to the at least one light emitting unit 31 mounted on the flexible circuit board 30.

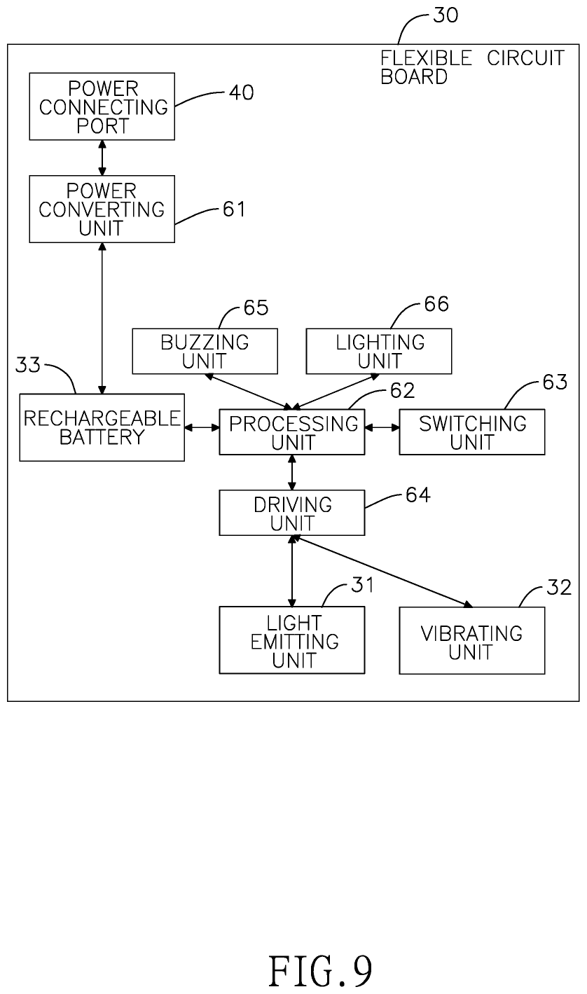

Moreover, with reference to FIG. 9, in a second embodiment of the present invention, the intraoral phototherapy device further includes a power connecting port 40, a power converting unit 61, a processing unit 62, a switching unit 63, a driving unit 64, a buzzing unit 65, a lighting unit 66, and a rechargeable battery 33.

In this embodiment, the power connecting port 40 is mounted on the transparent shell 10. The power converting unit 61 is electrically connected to the power connecting port 40 for receiving an external electricity provided by an external power, and the power converting unit 61 converts the external electricity to a powering electricity. The rechargeable battery 33 is electrically connected to the power converting unit 61 for receiving and storing the powering electricity. The processing unit 62 is electrically connected to the rechargeable battery 33 for receiving the powering electricity through the rechargeable battery 33. In this embodiment, the power connecting port 40 may be a USB female connecting port for inserting a charging wire, and for connecting to a power adaptor through the charging wire.

The switching unit 63 is electrically connected to the processing unit 62. When the switching unit 63 generates a starting signal, the processing unit 62 receives the starting signal, and generates a light controlling signal according to the starting signal.

The driving unit 64 is electrically connected to the processing unit 62. When the driving unit 64 receives the light controlling signal, the driving unit 64 generates a light driving signal according to the light controlling signal. When the driving unit 64 generates the light driving signal, the at least one light emitting unit 31 receives the light driving signal, and emits the light according to the light driving signal.

In this embodiment, the flexible circuit board 30 may further include at least one vibrating unit 32, and the at least one vibrating unit 32 is mounted on the light emitting surface 301 of the flexible circuit board 30. When the switching unit 63 generates the starting signal, the processing unit 62 receives the starting signal, and further generates a vibration controlling signal according to the starting signal. When the driving unit 64 receives the vibration controlling signal, the driving unit 64 generates a vibration driving signal according to the vibration controlling signal. When the driving unit 64 generates the vibration driving signal, the at least one vibrating unit 32 receives the vibration driving signal, and generates vibrations according to the vibration driving signal.

The second embodiment of the present invention mounts the rechargeable battery 33 in the transparent shell 10. Therefore, the intraoral phototherapy device may not need to connect to the external power through a connecting wire, and the intraoral phototherapy device can be integrated as an independent device without the connecting wire. Namely, when the user uses the intraoral phototherapy device, the intraoral phototherapy device can be directly inserted into an oral cavity of the user without opening a mouth of the user to allow the connecting wire to extend out, so that the discomforts of the user can be further soothed.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. An intraoral phototherapy device, comprising:
a transparent shell, comprising a bite portion and a light portion; wherein the light portion comprises a light surface and a back surface; wherein the bite portion is mounted on the light surface of the light portion;
a supporting board, mounted in the light portion of the transparent shell, and comprising a circuit mounting surface; wherein the circuit mounting surface faces to the light surface of the light portion;
a flexible circuit board, mounted on the circuit mounting surface of the supporting board, and comprising a light emitting surface; wherein the light emitting surface faces to the light surface of the light portion;
wherein the flexible circuit board further comprises:
at least one light emitting unit, mounted on the light emitting surface; wherein the at least one light emitting unit emits a light having wavelength of 600-1000 nanometers (nm).

2. The intraoral phototherapy device as claimed in claim 1, further comprising:
a power connecting port;
a first connecting wire;
a control box, comprising:
a power converting unit, connecting to the power connecting port through the first connecting wire; wherein the power converting unit receives an external electricity provided by an external power, and converts the external electricity to a powering electricity;
a processing unit, electrically connected to the power converting unit for receiving the powering electricity;
a switching unit, electrically connected to the processing unit; wherein when the switching unit generates a starting signal, the processing unit receives the starting signal, and generates a light controlling signal according to the starting signal;
a driving unit, electrically connected to the processing unit; wherein when the driving unit receives the light controlling signal, the driving unit generates a light driving signal according to the light controlling signal;
a second connecting wire, electrically connected between the at least one light emitting unit mounted on the flexible circuit board and the driving unit of the control box;
wherein when the driving unit generates the light driving signal, the at least one light emitting unit receives the light driving signal through the second connecting wire, and emits the light according to the light driving signal.

3. The intraoral phototherapy device as claimed in claim 2, wherein the flexible circuit board further comprises:
at least one vibrating unit, mounted on the light emitting surface of the flexible circuit board;
wherein when the switching unit generates the starting signal, the processing unit receives the starting signal, and further generates a vibration controlling signal according to the starting signal;
wherein when the driving unit receives the vibration controlling signal, the driving unit generates a vibration driving signal according to the vibration controlling signal;
wherein when the driving unit generates the vibration driving signal, the at least one vibrating unit receives the vibration driving signal through the second connecting wire, and generates vibrations according the vibration driving signal.

4. The intraoral phototherapy device as claimed in claim 2, wherein the control box further comprises:
a buzzing unit, electrically connected to the processing unit;
wherein when the switching unit generates the starting signal, the processing unit receives the starting signal, and further generates a reminding signal according to the starting signal;
wherein when the buzzing unit receives the reminding signal, the buzzing unit generates a reminding audio according to the reminding signal.

5. The intraoral phototherapy device as claimed in claim 2, wherein the control box further comprises:
a lighting unit, electrically connected to the processing unit;
wherein when the switching unit generates the starting signal, the processing unit receives the starting signal, and further generates the reminding signal according to the starting signal;
wherein when the lighting unit receives the reminding signal, the lighting unit emits a reminding light according to the reminding signal.

6. The intraoral phototherapy device as claimed in claim 1, wherein the control box further comprises:
a power connecting port, mounted on the transparent shell;
a power converting unit, electrically connected to the power connecting port for receiving an external electricity provided by an external power; wherein the power converting unit converts the external electricity to a powering electricity;
a rechargeable battery, electrically connected to the power converting unit for receiving and storing the powering electricity;
a processing unit, electrically connected to the rechargeable battery for receiving the powering electricity through the rechargeable battery;
a switching unit, electrically connected to the processing unit; wherein when the switching unit generates a starting signal, the processing unit receives the starting signal, and generates a light controlling signal according to the starting signal;
a driving unit, electrically connected to the processing unit; wherein when the driving unit receives the light controlling signal, the driving unit generates a light driving signal according to the light controlling signal;
wherein when the driving unit generates the light driving signal, the at least one light emitting unit receives the light driving signal, and emits the light according the light driving signal.

7. The intraoral phototherapy device as claimed in claim 1, wherein the bite portion of the transparent shell is flat, and has a first surface and a second surface opposite to the first surface;
wherein the first surface and the second surface of the bite portion are respectively perpendicular to the light surface of the light portion.

8. The intraoral phototherapy device as claimed in claim 7, wherein the first surface of the bite portion protrudes a positioning block.

9. The intraoral phototherapy device as claimed in claim 1, wherein the light portion of the transparent shell is an arc plate, and the light surface is opposite to the back surface;
wherein the light surface is an inner arc surface of the light portion.

10. The intraoral phototherapy device as claimed in claim 2, wherein the second connecting wire extends from the back surface of the light portion into the transparent shell to electrically connect to the at least one light emitting unit mounted on the flexible circuit board.
